(19) United States
(12) Patent Application Publication
(10) Pub. No.: US 2021/0093561 A1
(43) Pub. Date: Apr. 1, 2021

(54) DENTAL CARE CANNABIS DEVICE AND USE THEREOF

(71) Applicant: BUZZELET DEVELOPMENT AND TECHNOLOGIES LTD, Or-Akiva (IL)

(72) Inventors: Dana Berneman Zeitouni, Kfar Yona (IL); Aharon M. Eyal, Jerusalem (IL)

(21) Appl. No.: 17/120,197

(22) Filed: Dec. 13, 2020

Related U.S. Application Data
(63) Continuation-in-part of application No. PCT/IB2019/054928, filed on Jun. 12, 2019.
(60) Provisional application No. 62/683,956, filed on Jun. 12, 2018.

(51) Int. Cl. A61K 9/00; A61Q 11/00; A61K 8/49; A61K 31/352; A61K 8/34; A61K 31/05; A61K 31/015; A61K 8/31; A61K 31/01; A61K 31/336; A61K 31/045; A61K 8/35; A61K 31/122; A61K 31/575; A61K 8/63; A61K 31/085; A61K 31/231; A61K 8/37; A61K 36/315; A61K 8/9789; A61K 36/22; A61K 36/537; A61K 36/28; A61K 36/71; A61K 36/718; A61K 36/58; A61K 36/484; A61K 36/328; A61K 8/02

(52) U.S. Cl. CPC A61K 9/0053; A61K 2236/30; A61Q 11/00; A61K 8/498; A61K 31/352; A61K 8/347; A61K 31/05; A61K 31/015; A61K 8/31; A61K 31/01; A61K 31/336; A61K 8/4973; A61K 8/34; A61K 31/045; A61K 8/342; A61K 8/35; A61K 31/122; A61K 31/575; A61K 8/63; A61K 31/085; A61K 31/231; A61K 8/37; A61K 36/315; A61K 8/9789; A61K 36/22; A61K 36/537; A61K 36/28; A61K 36/71; A61K 36/718; A61K 36/58; A61K 36/484; A61K 36/328; A61K 8/0204; A61K 9/0063

(57) ABSTRACT

Provided is a device suitable for direct topical administration of a pharmaceutical composition comprising at least one active component selected from the group consisting of cannabinoids, terpenes, flavonoids, CB1 receptor agonists, CB2 receptor agonists and combinations thereof to a region of the oral cavity of a subject and methods of use thereof. The device comprises a solid substrate having at least a portion configured and dimensioned to be contacted with the region of the oral cavity of the subject; and an amount of the pharmaceutical composition associated with at least a portion of the solid substrate, wherein the amount of pharmaceutical composition is at least partially releasable from the portion of solid substrate.

### CROSS-REFERENCE TO RELATED APPLICATIONS

This patent application is a Continuation-in-Part (CIP) of PCT international application number PCT/IB2019/054928, having an international filing date of Jun. 12, 2019, published as international publication number WO 2019/139357 A1, which is hereby incorporated by reference in its entirety; which claims benefit and priority from U.S. 62/683,956, filed on Jun. 12, 2018, which is hereby incorporated by reference in its entirety.

### FIELD OF THE INVENTION

The field of art to which this invention generally pertains is dental care devices, and specifically to dental care devices for direct topical administration of a pharmaceutical composition comprising at least one active component selected from the group consisting of cannabinoids, terpenes, flavonoids, CB1 receptor agonists, CB2 receptor agonists and combinations thereof to a region of the oral cavity of a subject.

### BACKGROUND OF THE INVENTION

There is an ongoing need for improved and more effective treatment of conditions and/or symptoms associated with aggressive and chronic gum disease, gum pus pockets, gum inflammation, gum infection, gingivitis, dental plaque, periodontal, necrotizing periodontal disease, gum fungal infection, gum viral infection, bleeding gum, swollen gum, loosen teeth and plaque below gum line. There is further an ongoing need for improved and more effective treatment of conditions and/or symptoms associated with ulcer, viral infection, bacterial infection, fungal infection, infectious disease, inflammation, dermatophytes, periodontal bacteria and gum bacterial infection.

### SUMMARY OF THE INVENTION

According to an aspect of some embodiments of the invention, there is provided a device suitable for direct topical administration of a pharmaceutical composition comprising at least one active component selected from the group consisting of cannabinoids, terpenes, flavonoids, CB1 receptor agonists, CB2 receptor agonists and combinations thereof to a region of the oral cavity of a subject, the device comprising:

a solid substrate having at least a portion configured and dimensioned to be contacted with the region of the oral cavity of the subject; and

an amount of the pharmaceutical composition associated with at least a portion of the solid substrate, wherein the amount of pharmaceutical composition is at least partially releasable from the portion of the solid substrate,

wherein the solid substrate and the at least partially releasable association are such to allow direct administration of a pharmaceutically effective amount of the pharmaceutical composition to at least a portion of the region of the oral cavity of the subject when contacted therewith.

According to an embodiment, the substrate has a transverse cross-section of less than 5 millimeters.

According to an embodiment, an average amount of the active component of the pharmaceutical composition releasably associated with the at least a portion of substrate is at least 1 milligram per 100 meter length of substrate.

According to an embodiment, the pharmaceutical composition is associated with said at least a portion of the substrate, wherein the association is selected from at least one member of the group consisting of: the composition is absorbed in at least a portion of the substrate; the composition is adsorbed to at least a portion of the substrate; the substrate is coated with the composition; the substrate is impregnated with the composition; and the substrate is laminated with the composition.

According to an embodiment, at least 1% by weight of the pharmaceutical composition is releasable during contact with a surface of the region of the oral cavity.

According to an embodiment, the region of the oral cavity is selected from the group consisting of teeth, gums, palate and tongue.

According to an embodiment, the pharmaceutical composition further comprises at least one other active compound.

According to an embodiment, the pharmaceutical composition further comprises at least one herbal preparation, wherein the at least one herbal preparation is produced by at least one of distilling and extracting a portion of a plant, and wherein said plant is not Cannabis sativa.

According to an embodiment, the at least one herbal preparation is selected from the group consisting of extracts of Acorus calamus, Aloe spp, Ammi visnaga, Angelica archangelica Radix, Angelica sinensis, Artemisia absinthium, Asphalentum ponjabianum -mumio, Astragalus membranaceus, Atractylodes macrocephala, Avena sativa, Bacopa monnieri, Barosma betulina, Boswellia, Boswellia spp, Capsicum spp, Caulophyllum thalictroides, Centella (hydrocotyl) asisatica, Cimicifuga (Actaea) racemose, Cinnamomum zeylanicum, Coptis chinensis, Corydalis yanhusuo, Crataegus spp, Curcuma longa, Dioscorea Villosa spp, Echinacea spp, Eleutherococcus senticosus, Filipendula ulmaria, Foeniculum vulgare, Fucus vesiculosus, Galium aparine, Ganoderma lucidum, Garcinia cambogia, Ginkgo Biloba, Glycyrrhiza glabra, Grifola frondosa, Gymnema sylvestre, Harpagophytum procumbens, Humulus lupulus, Hydrastis canadensis, Hypericum perforatum, Iris versicolor, Laminaria spp, Lavendula officinalis, Lentinula edodes, Leonurus spp, Lepidium meyenii, lycium barbarum, Matricaria recutita, Melissa officinalis, Mentha piperita, Olea europea, origanum, Paeonia lactiflora, panax ginseng, Passiflora incarnata, Paullinia cupana, Paeonia lactiflora, Peumus boldus, Phelodendron amurense, Piscidia erythrina, Rehmania glutinosa, Rhamnus purshiana, Rhodiola rosea, Rosmarinus officinalis Rumex crispus, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum officinalis folia, Taraxacum spp. Radix, Tribulus terrestris, Trifolium pratense, Trigonella foenum graecum, Turnera diffusa, Uncaria tomentosa, Valeriana officinalis, Valleriane, Verbena officinalis, Viburnum opulus, Viburnum prunifolium, Viscum Album, Vitex agnus castus, Withinia somnifera, Zanthoxylum spp, Zingiber officinalis and combinations thereof.

According to an embodiment, the at least one herbal preparation is selected from the group consisting of Isatis tinctoria folia, pistacia lentiscus, Salvia officinalis, Calendual Officinalis, Echinacea spp., Hydrastis canadensis, Coptis chinensis, Azadirachta indica, Glycyrrhiza glabra and combinations thereof.

According to an embodiment, the at least one herbal preparation is selected from the group consisting of extracts of Glycyrrhiza glabra, salvia officinalis, Commiphora spp. and combinations thereof.

According to an embodiment, the pharmaceutical composition further comprises at least one excipient.

According to an embodiment, the substrate comprises at least one selected from the group consisting of fibers, threads and bristles and combinations thereof.

According to an embodiment, the fibers are selected from the group consisting of cannabis fibers, hemp fibers or combinations thereof.

According to an embodiment, the cannabinoids are selected from the group consisting of acid and/or decarboxylated forms of tetrahydrocannabinol (THC), cannabidiol (CBD), tetrahydrocannabivarin (THCV), cannabidivarin (CBDV), cannabigerol (CBG), cannabichromene (CBC), and cannabinol (CBN), cannabicyclol (CBL), cannabigerivarin (CBGV) and combinations thereof.

According to an embodiment, at least a fraction of the cannabinoids is obtained by a method selected from the group consisting of processing hemp, chemical synthesis, biological synthesis, isolation from plant source and combination thereof.

According to an embodiment, the terpenes are selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gujunene, farnesene, friedelin, carvacrol, eugenol, fenchol, geranyl acetate, camphor, menthol thymol and combinations thereof.

According to an embodiment, the terpenes comprise at least one terpene forming at least 40% by weight of the total amount of the terpenes.

According to an embodiment, the terpenes comprise at least one primary terpene selected from the group consisting of caryophyllene, pinene, limonene, eucalyptol, terpinene, terpineol, cymene, farnesene, guaiol, citronellol, humulene, terpinene, terpineol, nerolidol, Cycloartenol, caryophyllene oxide, amyrin, myrcene, linalool, phellandrene, eudesmol, thymol, carvacrol, eugenol, camphor, menthol, sabinene and combinations thereof.

According to an embodiment, the terpenes comprise at least one primary terpene selected from the group consisting of carvacol, eugenol, pinene, eucalyptol, amyrin and combinations thereof.

According to an embodiment, the terpenes comprise at least one primary terpene selected from the group consisting of caryophyllene, pinene, limonene, eucalyptol, terpinene, pinene, nerolidol, farnesene and combinations thereof.

According to an embodiment, the terpenes comprise at least one primary terpene selected from the group consisting of caryophyllene, pinene, limonene, eucalyptol, terpinene, pinene, nerolidol, farnesene and combinations thereof.

According to an embodiment, the terpenes comprise at least one primary terpene selected from the group consisting of myrcene, linalool, caryophyllene, eucalyptol, borneol, pinene, sabinene, terpineol, camphor, menthol and combinations thereof.

According to an embodiment, the composition further comprises a compound selected from the group of vitamin B complex, vitamin E, zinc, piperine and combinations thereof.

According to an embodiment, the device is selected from the group consisting of dental floss, a toothpick and a toothbrush.

According to an aspect of some embodiments of the present invention, there is provided a method for treating and/or preventing a condition of the oral cavity and/or a symptom associated therewith in a subject in need thereof, comprising topically applying the device of any one of the embodiments disclosed herein to a region of the oral cavity of the subject, wherein the region is selected from the group consisting of at least one of tooth, gums, palate and tongue, wherein said pharmaceutical composition is released onto a surface of the region.

According to an embodiment, the condition and/or symptom associated therewith is selected from the group consisting of aggressive and chronic gum disease, gum pus pockets, gum inflammation, gum infection, gingivitis, dental plaque, periodontal, necrotizing periodontal disease, gum fungal infection, gum viral infection, bleeding gum, swollen gum, loosen teeth, plaque below gum line, tooth decay, tooth erosion, tooth sensitivity, tongue infection, mouth sores, ulcer, viral infection, bacterial infection, fungal infection, infectious disease, inflammation, dermatophytes, periodontal bacteria, gum bacterial infection and combinations thereof.

According to an aspect of some embodiments of the present invention, there is provided a method for treating and/or preventing a condition of the oral cavity and/or a symptom associated therewith in a subject in need thereof, the method comprising:

providing a device suitable for direct topical administration of a pharmaceutical composition to the region of the oral cavity, the device comprising a solid substrate having at least a portion configured and dimensioned to be contacted with the region of the oral cavity and configured for releasable association with the pharmaceutical composition;

contacting at least a portion of the solid substrate with the pharmaceutical composition comprising at least one active component selected from the group consisting of cannabinoids, terpenes, flavonoids, CB1 receptor agonists, CB2 receptor agonists and combinations thereof, wherein an amount of the pharmaceutical composition releasably associates with the at least a portion of solid substrate; and

contacting the substrate releasably associated with the pharmaceutical composition with a surface of the region of the oral cavity, wherein the region is selected from the group consisting of at least one of tooth, gums, palate and tongue,

wherein the pharmaceutical composition is released onto a surface of the region, thereby treating and/or preventing the condition of the oral cavity and/or a symptom associated therewith.

According to an embodiment, contacting at least a portion of the solid substrate with the pharmaceutical composition comprises inserting the solid substrate into a container containing the pharmaceutical composition.

### BRIEF DESCRIPTION OF THE DRAWINGS

Some embodiments of the invention are described herein with reference to the accompanying figures. The description, together with the figures, makes apparent to a person having ordinary skill in the art how some embodiments of the invention may be practiced. The figures are for the purpose of illustrative discussion and no attempt is made to show structural details of an embodiment in more detail than is necessary for a fundamental understanding of the invention. For the sake of clarity, some objects depicted in the figures are not to scale.

In the Figures:

FIG. 1 is a perspective view of an embodiment of a device according to the principles of the present invention, wherein the device comprises a toothpick;

FIG. 2 is a perspective view of an alternative embodiment of a device according to the principles of the present invention, wherein the device comprises dental floss; and

FIG. 3 is a perspective view of an alternative embodiment of a device according to the principles of the present invention, wherein the device comprises a toothbrush.

### DETAILED DESCRIPTION OF THE INVENTION

As used herein the term ‘treating’ includes preventing, curing, ameliorating, mitigating and reducing the instances or severity of a condition or a symptom associated therewith.

As used herein, the term ‘substrate’ means any elongated solid structure, including flexible, partially flexible and non-flexible structures.

Unless indicated otherwise, percent is weight percent and ratio is weight/weight ratio. Unless indicated otherwise, weight ratio means the ratio between weight content, e.g. in an aqueous solution containing 20% solute and 80% water, the solute to water weight ratio is 20:80 or 1:4.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

According to an aspect of some embodiments of the invention, there is provided a device suitable for direct topical administration of a pharmaceutical composition comprising at least one active component selected from the group consisting of cannabinoids, terpenes, flavonoids, CB1 receptor agonists, CB2 receptor agonists and combinations thereof to a region of the oral cavity of a subject, the device comprising:

a solid substrate having at least a portion configured and dimensioned to be contacted with the region of the oral cavity of the subject; and

an amount of the pharmaceutical composition associated with at least a portion of the solid substrate, wherein the amount of pharmaceutical composition is at least partially releasable from the portion of the solid substrate,

wherein the solid substrate and the at least partially releasable association are such to allow direct administration of a pharmaceutically effective amount of the pharmaceutical composition to at least a portion of the region of the oral cavity of the subject when contacted therewith.

According to an embodiment, the substrate has a transverse cross-section of less than 5 millimeters, such as, for example less than 5 millimeters, less than 4 millimeters, less than 3 millimeters or less than 2 millimeters.

According to an embodiment, the transverse cross-section is of an elliptical shape, such as, for example, circular or oval. According to an embodiment, the transverse cross-section is in the shape of a quadrilateral, such as, for example, square or rectangular.

According to an embodiment, an average amount of the active component of the pharmaceutical composition releasably associated with the at least a portion of substrate is at least 1 milligram per 100 meter length of substrate. According to some embodiments, an average amount of the active component of the pharmaceutical composition releasably associated with the at least a portion of substrate is at least 1 milligram per 50 meter length of substrate, per 40 meter length of substrate, per 30 meter length of substrate, per 20 meter length of substrate or per 10 meter length of substrate.

According to an embodiment, upon release of the pharmaceutical composition in the region of the oral cavity of the subject, the active component is initially provided at a concentration of about 15-75 mg/L saliva.

According to an embodiment, the pharmaceutical composition is associated with said at least a portion of the substrate, wherein the association is selected from at least one member of the group consisting of: the composition is absorbed in at least a portion of the substrate; the composition is adsorbed to at least a portion of the substrate; the substrate is coated with the composition; the substrate is impregnated with the composition; and the substrate is laminated with the composition.

According to an embodiment, the pharmaceutical composition is contained within capsules and the capsules are releasably associated with the substrate.

According to an embodiment, at least 1% by weight of the pharmaceutical composition is releasable during contact with a surface of the region of the oral cavity, such as at least 1%, at least 10%, at least 20%, at least 30%, at least 40%, at least 50% or at least 50% by weight.

According to an embodiment, the region of the oral cavity is selected from the group consisting of teeth, gums, palate and tongue.

According to an embodiment, the pharmaceutical composition further comprises at least one other active compound.

According to an embodiment, the pharmaceutical composition comprises at least two active components, at least three active components, at least four active components or at least five active components.

According to an embodiment, the pharmaceutical composition further comprises at least one herbal preparation, wherein the at least one herbal preparation is produced by at least one of distilling and extracting a portion of a plant, and wherein said plant is not Cannabis sativa.

According to an embodiment, the at least one herbal preparation is selected from the group consisting of extracts of Acorus calamus, Aloe spp, Ammi visnaga, Angelica archangelica Radix, Angelica sinensis, Artemisia absinthium, Asphalentum ponjabianum-mumio, Astragalus membranaceus, Atractylodes macrocephala, Avena sativa, Bacopa monnieri, Barosma betulina, Boswellia, Boswellia spp, Capsicum spp, Caulophyllum thalictroides, Centella (hydrocotyl) asisatica, Cimicifuga (Actaea) racemose, Cinnamomum zeylanicum, Coptis chinensis, Corydalis yanhusuo, Crataegus spp, Curcuma longa, Dioscorea Villosa spp, Echinacea spp, Eleutherococcus senticosus, Filipendula ulmaria, Foeniculum vulgare, Fucus vesiculosus, Galium aparine, Ganoderma lucidum, Garcinia cambogia, Ginkgo Biloba, Glycyrrhiza glabra, Grifola frondosa, Gymnema sylvestre, Harpagophytum procumbens, Humulus lupulus, Hydrastis canadensis, Hypericum perforatum, Iris versicolor, Laminaria spp, Lavendula officinalis, Lentinula edodes, Leonurus spp, Lepidium meyenii, lycium barbarum, Matricaria recutita, Melissa officinalis, Mentha piperita, Olea europea, origanum, Paeonia lactiflora, panax ginseng, Passiflora incarnata, Paullinia cupana, Paeonia lactiflora, Peumus boldus, Phelodendron amurense, Piscidia erythrina, Rehmania glutinosa, Rhamnus purshiana, Rhodiola rosea, Rosmarinus officinalis Rumex crispus, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum officinalis folia, Taraxacum spp. Radix, Tribulus terrestris, Trifolium pratense, Trigonella foenum graecum, Turnera diffusa, Uncaria tomentosa, Valeriana officinalis, Valleriane, Verbena officinalis, Viburnum opulus, Viburnum prunifolium, Viscum Album, Vitex agnus castus, Withinia somnifera, Zanthoxylum spp, Zingiber officinalis and combinations thereof.

According to an embodiment, the at least one herbal preparation is selected from the group consisting of an extract of a plant selected from the group consisting of Isatis tinctoria folia, pistacia lentiscus, Salvia officinalis, Calendual Officinalis, Echinacea spp., Hydrastis canadensis, Coptis chinensis, Azadirachta indica, Glycyrrhiza glabra and combinations thereof.

According to an embodiment, the at least one herbal preparation is selected from the group consisting of extracts of Glycyrrhiza glabra, salvia officinalis, Commiphora spp. and combinations thereof.

According to an embodiment, the pharmaceutical composition further comprises at least one excipient, such as, for example, a wax.

According to an embodiment, the substrate comprises artificial and/or natural materials. According to some embodiments, the substrate comprises a material selected from the group consisting of cotton, cannabis, hemp, plastic, wood, metal, bamboo or combination thereof.

According to an embodiment, the substrate comprises at least one selected from the group consisting of fibers, threads and bristles and combinations thereof.

According to an embodiment, the fibers are selected from the group consisting of cannabis fibers, hemp fibers or combinations thereof.

According to an embodiment, the active component is derived from a cannabis plant. According to some such embodiments, the active component comprises an extract of cannabis matter. According to some such embodiments, the active component comprises an extract of cannabis inflorescence.

According to an embodiment, the active component comprises at least one cannabinoid selected from the group consisting of a synthetic cannabinoid, a natural cannabinoid, a derivative of a natural cannabinoid, or combinations thereof.

According to an embodiment, the at least one cannabinoid is provided in its acid form. According to an embodiment, the at least one cannabinoid is provided in decarboxylated form.

According to an embodiment, the cannabinoids are selected from the group consisting of acid and/or decarboxylated forms of tetrahydrocannabinol (THC), cannabidiol (CBD), tetrahydrocannabivarin (THCV), cannabidivarin (CBDV), cannabigerol (CBG), cannabichromene (CBC), and cannabinol (CBN), cannabicyclol (CBL), cannabigerivarin (CBGV) and combinations thereof.

According to an embodiment, at least a fraction of the cannabinoids is obtained by a method selected from the group consisting of processing hemp, chemical synthesis, biological synthesis, isolation from plant source and combination thereof.

According to an embodiment, the active component terpenes comprise at least one terpene selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gujunene, farnesene, friedelin, carvacrol, eugenol, fenchol, geranyl acetate, camphor, menthol and combinations thereof.

According to an embodiment, the terpenes comprise at least one terpene forming at least 40% by weight of the total amount of the total terpenes, such as at least 40%, at least 45%, at least 50%, at least 55%, at least 60%, at least 65% or at least 70% by weight of the total amount of the total terpenes.

According to an embodiment, the terpenes comprise at least one primary terpene selected from the group consisting of caryophyllene, pinene, limonene, eucalyptol, terpinene, terpineol, cymene, farnesene, guaiol, citronellol, humulene, terpinene, terpineol, nerolidol, Cycloartenol, caryophyllene oxide, amyrin, myrcene, linalool, phellandrene, eudesmol, thymol, carvacrol, eugenol, camphor, menthol, sabinene and combinations thereof.

According to an embodiment, the terpenes comprise at least one primary terpene selected from the group consisting of carvacol, eugenol, pinene, eucalyptol, amyrin and combinations thereof.

According to an embodiment, the terpenes comprise at least one primary terpene selected from the group consisting of caryophyllene, pinene, limonene, eucalyptol, terpinene, pinene, nerolidol, farnesene and combinations thereof.

According to an embodiment, the terpenes comprise at least one primary terpene selected from the group consisting of caryophyllene, pinene, limonene, eucalyptol, terpinene, pinene, nerolidol, farnesene and combinations thereof.

According to an embodiment, the terpenes comprise at least one primary terpene selected from the group consisting of myrcene, linalool, caryophyllene, eucalyptol, borneol, pinene, sabinene, terpineol, camphor, menthol and combinations thereof.

According to an embodiment, the pharmaceutical composition further comprises a compound selected from the group of vitamin B complex, vitamin E, zinc, piperine and combinations thereof.

According to an embodiment, the device is a dental device selected from the group consisting of dental floss, a toothpick and a toothbrush.

According to an aspect of some embodiments of the present invention, there is provided a method for treating and/or preventing a condition of the oral cavity and/or a symptom associated therewith in a subject in need thereof, comprising topically applying the pharmaceutical composition associated with the substrate of the device of any one of the embodiments disclosed herein to a region of the oral cavity of the subject, wherein the region is selected from the group consisting of at least one of tooth, gums, palate and tongue, wherein said pharmaceutical composition is released onto a surface of the region.

According to an embodiment, the condition and/or symptom associated therewith is selected from the group consisting of aggressive and chronic gum disease, gum pus pockets, gum inflammation, gum infection, gingivitis, dental plaque, periodontal, necrotizing periodontal disease, gum fungal infection, gum viral infection, bleeding gum, swollen gum, loosen teeth, plaque below gum line, tooth decay, tooth erosion, tooth sensitivity, tongue infection, mouth sores and combinations thereof.

According to an embodiment, the condition and/or symptom associated therewith is selected from the group consisting of ulcer, viral infection, bacterial infection, fungal infection, infectious disease, inflammation, dermatophytes, periodontal bacteria, gum bacterial infection and combinations thereof.

According to an embodiment, the device for use in the method disclosed herein is a dental device selected from the group consisting of dental floss, a toothpick and a toothbrush.

According to an embodiment, the method comprises contacting the surface of the region of the oral cavity to be treated with the device as disclosed herein in the form of a dental floss. According to an embodiment, the method comprises contacting the surface of the region of the oral cavity to be treated with the device as disclosed herein in the form of a toothpick. According to an embodiment, the method comprises contacting the surface of the region of the oral cavity to be treated by brushing with the device as disclosed herein in the form of a toothbrush.

According to an embodiment, the method as disclosed herein provides an enhanced therapeutic effect compared to similar dental treatment using a similar device comprising a similar substrate in the absence of the pharmaceutical composition comprising an active component as disclosed herein. According to an embodiment, said enhanced therapeutic effect comprises a shortened onset time, an increased magnitude, an extended duration, reduced dosages, reduced secondary adverse symptoms, reduced frequency of conditions and/or symptoms, reduced severity of conditions and/or symptoms, reduced consumption of other drugs and combinations thereof.

According to an aspect of some embodiments of the present invention, there is provided a method for treating and/or preventing a condition of the oral cavity and/or a symptom associated therewith in a subject in need thereof, the method comprising:

providing a device suitable for direct topical administration of a pharmaceutical composition to the region of the oral cavity, the device comprising a solid substrate having at least a portion configured and dimensioned to be contacted with the region of the oral cavity and configured for releasable association with the pharmaceutical composition;

contacting at least a portion of the solid substrate with the pharmaceutical composition comprising at least one active component selected from the group consisting of cannabinoids, terpenes, flavonoids, CB1 receptor agonists, CB2 receptor agonists and combinations thereof, wherein an amount of the pharmaceutical composition releasably associates with the at least a portion of solid substrate; and

contacting the substrate releasably associated with the pharmaceutical composition with a surface of the region of the oral cavity, wherein the region is selected from the group consisting of at least one of tooth, gums, palate and tongue,

wherein the pharmaceutical composition is released onto a surface of the region, thereby treating and/or preventing the condition of the oral cavity and/or a symptom associated therewith.

According to an embodiment, contacting at least a portion of the solid substrate with the pharmaceutical composition comprises inserting the solid substrate into a container containing the pharmaceutical composition.

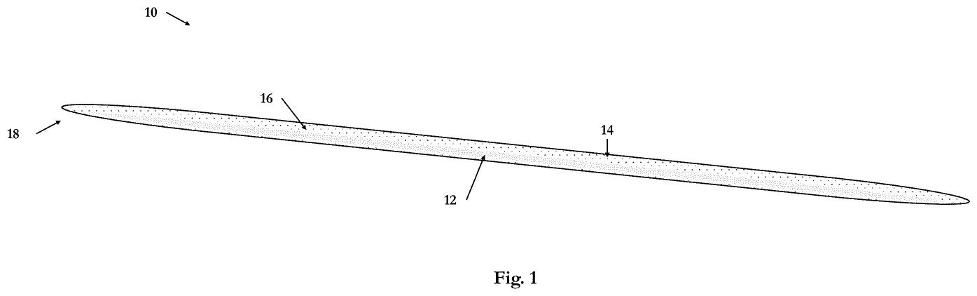

Referring now to FIG. 1, there is shown a perspective view of an embodiment of the device of the present invention, comprising a toothpick 10 comprising a substrate 12 formed of a suitable material such as wood, wherein at least a portion of substrate 12 is releasably associated with a pharmaceutical composition 14 comprising a water-soluble adhesive as carrier and at least one active component selected from the group consisting of cannabinoids, terpenes, flavonoids, CB1 receptor agonists, CB2 receptor agonists and combinations thereof in the form of a coating 16. Toothpick 10 is dimensioned to be insertable into a space between two adjacent teeth in order to contact a portion of the gum between the two adjacent teeth. Toothpick 10 may be rigid or semi-rigid. Contacting the surface of the gum to be treated, with coating 16 of substrate 12, with concurrent application of moderate pressure to toothpick 10, causes dissolution of the carrier and thereby release of pharmaceutical composition 14 at the treatment site. Toothpick 10 optionally has at least one tapered end (the distal end) 18 to increase the ease of insertion of toothpick 10 into the region of the treatment site.

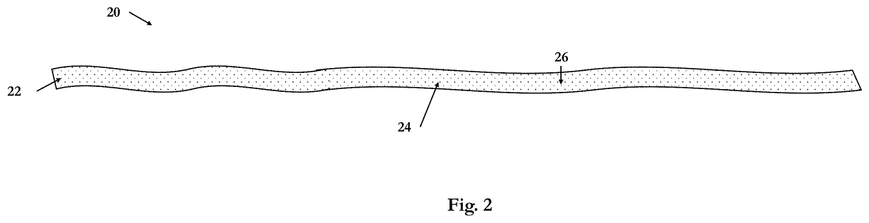

FIG. 2, shows a perspective view of an alternative embodiment of the device of the present invention, comprising a length of dental floss 20 formed from a cotton fiber 22 wherein at least a section along the length of dental floss 20 comprises a substrate 24 impregnated with a pharmaceutical composition 14 comprising at least one active component selected from the group consisting of cannabinoids, terpenes, flavonoids, CB1 receptor agonists, CB2 receptor agonists and combinations thereof, in some embodiments also with a water-soluble adhesive as a carrier. As known in the art, dental floss 20 is dimensioned to be insertable into a space between two adjacent teeth in order to contact a portion of the gum between the two adjacent teeth. Contacting the surface of the gum to be treated, with substrate 24, with concurrent application of moderate pressure, such as by moving dental floss 20 backwards and forwards against the surface of the gum as in the conventional use of dental floss, causes release of pharmaceutical composition 14 at the treatment site.

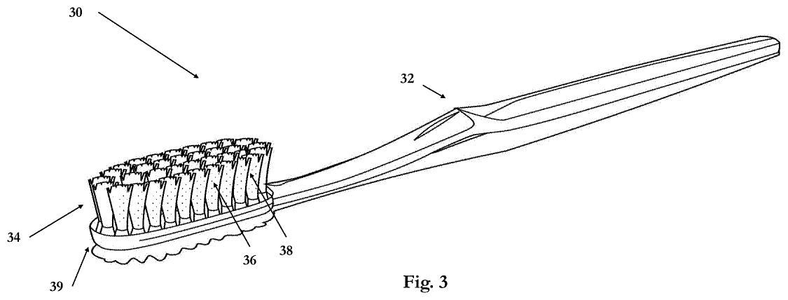

FIG. 3, shows a perspective view of an alternative embodiment of the device of the present invention, comprising a toothbrush 30 comprising a handle 32 and a plurality of substrates in the form of bristles 34 (made, for example, of bamboo viscose) extending from distal end 36 of handle 32 substantially perpendicular to the longitudinal axis of handle 34. A pharmaceutical composition 14 comprising at least one active component selected from the group consisting of cannabinoids, terpenes, flavonoids, CB1 receptor agonists, CB2 receptor agonists and combinations thereof is adsorbed to the outer surface 38 of bristles 34. Bristles 34 are dimensioned to be insertable into a space between two adjacent teeth in order to contact a portion of the gum between the two adjacent teeth. Contacting the surface of the gum to be treated, with bristles 34, with concurrent application of moderate pressure, such as by the normal pressure applied during conventional use of a toothbrush, causes release of the pharmaceutical composition at the treatment site.

1. A device suitable for direct topical administration of a pharmaceutical composition comprising at least one active component selected from the group consisting of cannabinoids, terpenes, flavonoids, CB1 receptor agonists, CB2 receptor agonists and combinations thereof to a region of the oral cavity of a subject, the device comprising:
a solid substrate having at least a portion configured and dimensioned to be contacted with the region of the oral cavity of the subject; and
an amount of the pharmaceutical composition associated with at least a portion of said solid substrate, wherein said amount of pharmaceutical composition is at least partially releasable from said portion of said solid substrate,
wherein said solid substrate and said at least partially releasable association are such to allow direct administration of a pharmaceutically effective amount of said pharmaceutical composition to at least a portion of said region of the oral cavity of the subject when contacted therewith.

2. The device of claim 1, wherein said substrate has a transverse cross-section of less than 5 millimeters.

3. The device of claim 1, wherein an average amount of said active component of said pharmaceutical composition releasably associated with said at least a portion of said substrate is at least 1 milligram per 100 meter length of said substrate.

4. The device of claim 1, wherein said pharmaceutical composition is associated with said at least a portion of said substrate, wherein said association is selected from at least one member of the group consisting of: said composition is absorbed in at least a portion of said substrate; said composition is adsorbed to at least a portion of said substrate; said substrate is coated with said composition; said substrate is impregnated with said composition; and said substrate is laminated with said composition

5. The device of claim 1, wherein at least 1% by weight of said pharmaceutical composition is releasable during contact with a surface of said region of said oral cavity.

6. The device of claim 5, wherein said region of said oral cavity is selected from the group consisting of teeth, gums, palate and tongue.

7. The device of claim 1, wherein said substrate comprises at least one selected from the group consisting of fibers, threads and bristles and combinations thereof.

8. The device of claim 7, wherein said fibers are selected from the group consisting of cannabis fibers, hemp fibers or combinations thereof.

9. The device of claim 1, wherein said cannabinoids are selected from the group consisting of acid and/or decarboxylated forms of tetrahydrocannabinol (THC), cannabidiol (CBD), tetrahydrocannabivarin (THCV), cannabidivarin (CBDV), cannabigerol (CBG), cannabichromene (CBC), and cannabinol (CBN), cannabicyclol (CBL), cannabigerivarin (CBGV) and combinations thereof.

10. The device of claim 1, wherein said terpenes are selected from the group consisting of pinene, limonene, linalool, caryophyllene, caryophyllene oxide, myrcene, humulene, borneol, eucalyptol, terpineol, nerolidol, phytol, geraniol, bisabolol, camphene, beta-amyrin, thujone, citronellol, pulegone, cycloartenol, cymene, sabinene, carene, terpinene, fenchol, isopulegol, guaiol, phellandrene, eudesmol, ocimene, cardinene, elemene, gujunene, farnesene, friedelin, carvacrol, eugenol, fenchol, geranyl acetate, camphor, menthol, thymol and combinations thereof.

11. The device of claim 1, wherein said pharmaceutical composition further comprises at least one herbal preparation, wherein said at least one herbal preparation is produced by at least one of distilling and extracting a portion of a plant, and wherein said plant is not Cannabis sativa.

12. The device of claim 1, wherein said terpenes comprise at least one terpene forming at least 40% by weight of the total amount of said terpenes.

13. The device of claim 11, wherein said at least one herbal preparation is selected from the group consisting of extracts of Acorus calamus, Aloe spp, Ammi visnaga, Angelica archangelica Radix, Angelica sinensis, Artemisia absinthium, Asphalentum ponjabianum-mumio, Astragalus membranaceus, Atractylodes macrocephala, Avena sativa, Bacopa monnieri, Barosma betulina, Boswellia, Boswellia spp, Capsicum spp, Caulophyllum thalictroides, Centella (hydrocotyl) asisatica, Cimicifuga (Actaea) racemose, Cinnamomum zeylanicum, Coptis chinensis, Corydalis yanhusuo, Crataegus spp, Curcuma longa, Dioscorea Villosa spp, Echinacea spp, Eleutherococcus senticosus, Filipendula ulmaria, Foeniculum vulgare, Fucus vesiculosus, Galium aparine, Ganoderma lucidum, Garcinia cambogia, Ginkgo Biloba, Glycyrrhiza glabra, Grifola frondosa, Gymnema sylvestre, Harpagophytum procumbens, Humulus lupulus, Hydrastis canadensis, Hypericum perforatum, Iris versicolor, Laminaria spp, Lavendula officinalis, Lentinula edodes, Leonurus spp, Lepidium meyenii, lycium barbarum, Matricaria recutita, Melissa officinalis, Mentha piperita, Olea europea, origanum, Paeonia lactiflora, panax ginseng, Passiflora incarnata, Paullinia cupana, Paeonia lactiflora, Peumus boldus, Phelodendron amurense, Piscidia erythrina, Rehmania glutinosa, Rhamnus purshiana, Rhodiola rosea, Rosmarinus officinalis Rumex crispus, Salix alba, Salvia miltiorrhiza, Salvia officinalis, Sambucus nigra, Schisandra chinensis, Scutalleria laterifolia, Scutellaria baicalensis, Silybum marianum, Sophora flavescens, Stachys betonica, Tabebuia impetiginosa, Tanacetum parthenium, Taraxacum officinalis folia, Taraxacum spp. Radix, Tribulus terrestris, Trifolium pratense, Trigonella foenum graecum, Turnera diffusa, Uncaria tomentosa, Valeriana officinalis, Valleriane, Verbena officinalis, Viburnum opulus, Viburnum prunifolium, Viscum Album, Vitex agnus castus, Withinia somnifera, Zanthoxylum spp, Zingiber officinalis and combinations thereof.

14. The device of claim 11, wherein said at least one herbal preparation is selected from the group consisting of an extract of a plant selected from the group consisting of Isatis tinctoria folia, pistacia lentiscus, Salvia officinalis, Calendual Officinalis, Echinacea spp., Hydrastis canadensis, Coptis chinensis, Azadirachta indica, Glycyrrhiza glabra and combinations thereof.

15. The device of claim 11, wherein said at least one herbal preparation is selected from the group consisting of extracts of Glycyrrhiza glabra, salvia officinalis, Commiphora spp. and combinations thereof.

16. The device of claim 1, selected from the group consisting of dental floss, a toothpick and a toothbrush.

17. A method for treating and/or preventing a condition of the oral cavity and/or a symptom associated therewith in a subject in need thereof, comprising topically applying the device of any one of claims 1 to 24 to a region of the oral cavity of the subject, wherein said region is selected from the group consisting of at least one of tooth, gums, palate and tongue, wherein said pharmaceutical composition is released onto a surface of said region.

18. The method of claim 17, wherein said condition and/or symptom associated therewith is selected from the group consisting of aggressive and chronic gum disease, gum pus pockets, gum inflammation, gum infection, gingivitis, dental plaque, periodontal, necrotizing periodontal disease, gum fungal infection, gum viral infection, bleeding gum, swollen gum, loosen teeth, plaque below gum line, tooth decay, tooth erosion, tooth sensitivity, tongue infection, mouth sores, ulcer, viral infection, bacterial infection, fungal infection, infectious disease, inflammation, dermatophytes, periodontal bacteria, gum bacterial infection and combinations thereof.

19. A method for treating and/or preventing a condition of the oral cavity and/or a symptom associated therewith in a subject in need thereof, the method comprising:
providing a device suitable for direct topical administration of a pharmaceutical composition to the region of the oral cavity, said device comprising a solid substrate having at least a portion configured and dimensioned to be contacted with the region of the oral cavity and configured for releasable association with said pharmaceutical composition;
contacting at least a portion of said solid substrate with said pharmaceutical composition comprising at least one active component selected from the group consisting of cannabinoids, terpenes, flavonoids, CB1 receptor agonists, CB2 receptor agonists and combinations thereof, wherein an amount of said pharmaceutical composition releasably associates with said at least a portion of said solid substrate; and
contacting said substrate releasably associated with said pharmaceutical composition with a surface of the region of the oral cavity, wherein the region is selected from the group consisting of at least one of tooth, gums, palate and tongue,
wherein said pharmaceutical composition is released onto a surface of the region, thereby treating and/or preventing the condition of the oral cavity and/or a symptom associated therewith.

20. The method of claim 19, wherein said contacting at least a portion of said solid substrate with said pharmaceutical composition comprises inserting said solid substrate into a container containing said pharmaceutical composition.
